(19) United States
(12) Patent Application Publication
(10) Pub. No.: US 2025/0032724 A1
(43) Pub. Date: Jan. 30, 2025

(54) INJECTION DEVICE AND METHOD OF UNLOCKING OPERATION OF THE INJECTION DEVICE

(71) Applicant: Sanofi, Paris (FR)

(72) Inventors: Stefan Alt, Frankfurt am Main (DE); Michael Helmer, Frankfurt am Main (DE); Michael Schabbach, Frankfurt am Main (DE); Martin Vitt, Frankfurt am Main (DE)

(21) Appl. No.: 18/706,854

(22) PCT Filed: Nov. 1, 2022
(86) PCT No.: PCT/EP2022/080405

§ 371 Date: May 2, 2024

(30) Foreign Application Priority Data
Nov. 3, 2021 (EP) 21315219.3

(51) Int. Cl. A61M 5/315

(52) U.S. Cl. CPC A61M 5/31546; A61M 5/31571

(57) ABSTRACT

The present disclosure relates to a method of unlocking operation of an injection device. The injection device includes a housing configured to accommodate a medicament container, and a drive mechanism operably engageable with the medicament container for expelling or withdrawing a dose of medicament from the medicament container. The drive mechanism includes at least one interlock configured to disable at least one of setting of the dose and dispensing of the dose. The injection device further includes an identifier provided on or associated with the medicament container, and an electronic unit operable to control the at least one interlock. The electronic unit includes a NFC (NFC) interface for obtaining information indicative of the identifier. The electronic unit is configured to process signals of the NFC interface to at least temporarily deactivate the at least one interlock.

### CROSS REFERENCE TO RELATED APPLICATIONS

The present application is the national stage entry of International Patent Application No. PCT/EP2022/080405, filed on Nov. 1, 2022, and claims priority to Application No. EP 21315209.3, filed on Nov. 3, 2021, the disclosures of which are incorporated herein by reference.

### TECHNICAL FIELD

The present disclosure relates to an injection device, such as a pen-type injector, and to a method of unlocking operation of the injection device, e.g., by reading or capturing of a passive identifier provided on or associated with a medicament container. In further aspects, the present disclosure relates to an electronic device, an injection system, drug delivery system, and computer-readable medium configured to implement unlocking of the injection device.

### BACKGROUND

Drug delivery devices for setting and dispensing a single or multiple doses of a liquid medicament are used for various purposes. Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore tend to be robust in construction and easy to use. Furthermore, manipulation and general handling of the device and components thereof should be intelligible and easily understood. It may be beneficial for such injection devices to provide setting and subsequent dispensing of a dose of a medicament of variable size. Moreover, a dose setting as well as a dose dispensing procedure should generally be easy to operate and unambiguous.

A patient suffering from a particular disease may require a certain amount of a medicament, which can either be injected via a pen-type injection syringe or infused via a pump.

Some drug delivery or injection devices provide selecting of a dose of a medicament of variable size and injecting a dose previously set. Other injection devices provide setting and dispensing of a fixed dose. In such cases, the amount of medicament that should be injected in accordance with a given prescription schedule does not change (or cannot be changed) over time.

Some injection devices are implemented as a reusable injection device with a replaceable medicament container, such as a cartridge. Other injection devices are implemented as a disposable injection device. With disposable injection devices, the entirety of the injection device is discarded when the content (e.g., the medicament) has been used.

With reusable injection devices a medicament container, e.g., a cartridge has to be replaced when the medicament has been depleted. Here, the medicament container, e.g., a vitreous cartridge filled with the medicament has to be replaced. Some primary medicament containers, e.g., in the form of a cartridge filled with the medicament are preassembled in a housing component of the injection device, such as a cartridge holder.

For certain reusable devices and disposable injection devices, only a dedicated medicament container or dedicated housing component preassembled with such a medicament container can be used with a dedicated drive mechanism of the injection device.

Generally, there is a large variety of primary medicament containers that differ with regard to their filling volume or pharmaceutical substance or concentration of the medicament. For replacement of a medicament container, an injection device or drive mechanism can only be used in conjunction with a dedicated medicament container intended for use with this particular type of injection device or drive mechanism. Unintended cross-use of non-matching pairs of medicament containers and drive mechanisms or injection devices should be avoided.

Some approaches involve mechanically encoding the primary container, e.g., the medicament container and/or the cartridge holder equipped with such a medicament container, to prevent unintended mechanical connection to a non-matching drive mechanism or non-matching injection device. Such solutions involve a redesign of respective mechanical components of the injection device. Providing a large variety of differently mechanically encoded housing components is rather elaborate in terms of manufacturing and manufacturing logistics of the injection device.

### SUMMARY

The present disclosure relates to an improved injection device, electronic device, injection system, method of operating an injection device, and computer-readable medium to enhance patient safety and prevent unintended cross-use of replaceable medicament containers with an injection device. The present disclosure aims to avoid a mechanical redesign of housing components of existing injection devices. The devices and techniques described herein are easily integrable with existing injection devices or injection systems, and adapt to varying demands or scenarios of use of the injection device.

In one aspect, the present disclosure relates to an injection device for injecting of a dose of a medicament. The injection device includes a housing configured to accommodate a medicament container. The injection device further includes a drive mechanism operably engageable with the medicament container for expelling or withdrawing the dose of the medicament from the medicament container. The drive mechanism includes at least one interlock configured to disable at least one of setting of the dose and dispensing of the dose of the medicament. With an activated interlock at least one of setting of the dose and dispensing of the dose is disabled. With a disabled interlock, setting and/or dispensing of the dose is enabled.

There is further provided an identifier on the medicament container or associated with the medicament container. Furthermore, the injection device includes an electronic unit. The electronic unit or electronic module is operable to control the at least one interlock. The injection device further includes a near-field communication (NFC) interface for obtaining information being indicative of the identifier or data provided by the identifier. The electronic unit is further configured to process signals of the NFC interface to at least temporarily deactivate the at least one interlock.

The electronic unit and/or the NFC interface is or are operable to read information directly from the identifier or to communicate with the identifier, when the identifier is provided on the medicament container or when the identifier is associated with the medicament container.

Information being indicative of the identifier and/or identifier information being indicative of the medicament container can be captured, read and/or processed by the electronic unit of the injection device. Based on the identifier related information retrievable by the NFC interface respective signals can be processed by the electronic unit for locking and/or unlocking the at least one interlock. Based on signals obtained from the NFC interface the at least one interlock can be at least temporarily deactivated.

When the information or data which is indicative of the identifier matches with predefined medicament container related data the respective medicament container can be used with the drive mechanism of the injection device.

In some embodiments, the electronic unit includes an electronic storage operable to store predefined medicament container related data. The predefined medicament container related data may be indicative of at least one or numerous particular medicament containers that can be used or should be used with the injection device. The predefined medicament container related data may be also indicative of particular medicament containers that should not be used with the injection device. The electronic storage of the electronic unit may be provided with the predefined medicament container related data upon manufacturing or assembly of the injection device. The electronic storage of the electronic unit may be implemented as a read-only memory (ROM).

With some embodiments, where the identifier does not match with the drive mechanism and/or where the information obtained by the NFC interface does not match with predefined medicament container related data the interlock remains activated and use of the injection device, e.g., for the purpose of setting and/or dispensing of the dose, is impeded or blocked.

The identifier can be implemented as an electronic identifier, as an electronic passive identifier, as an optical or visual identifier or the like. The identifier cooperates and matches with the NFC interface or with a respective sensor of the electronic unit.

The electronic unit can be embedded in or on the injection device, and may be arranged inside the housing of the injection device and may be permanently connected to or assembled inside the injection device. In some embodiments, the electronic unit is an embedded electronic unit being integrated into the injection device or drive mechanism thereof.

In some embodiments, the electronic unit includes a processor connected to the NFC interface and being operable to process the signals of the NFC interface. The at least one processor may be also operably connected to the at least one interlock and may be operable to control or to manipulate the interlock, e.g., so as to switch the interlock between an interlocked state and a release state. Deactivation of the interlock may correspond to switch the interlock into the release state. Activation of the interlock corresponds to a switching into the interlocked state.

The at least one processor of the electronic unit may be further operably connected with the electronic storage to read out the predefined medicament container related data.

The at least one processor is operable to compare the predefined medicament container related data as obtained from the electronic storage with information obtained by the NFC interface being indicative of a medicament container that is used or should be actually used with the injection device.

In some embodiments, the at least one processor is configured to operate in a stand-alone mode, and may be operable to process signals obtained from the NFC interface. Hence, the at least one processor may be operable to compare the signals from the NFC interface indicative of the type of medicament container currently in use with predefined medicament container related data e.g., provided by the electronic storage of the electronic unit. Such comparison may reveal if the medicament container currently in use is suitable for the injection device. Depending on this comparison the at least one processor may be further operable to activate or to deactivate the at least one interlock of the injection device.

With some embodiments the NFC interface is only able to read or to capture the identifier and to transmit the identifier information to the electronic unit, e.g., to a processor of the electronic unit for further signal processing. With further examples the NFC interface is able to modify the identifier, e.g., to store data in the identifier so as to mark the identifier of being read.

Signal processing may be conducted by the electronic unit of the injection device to keep the interlock activated, e.g., when the identifier does not match with a predefined requirement, demand or scenario of use. The electronic unit may keep the at least one interlock activated so that at least one of setting and dispensing of the dose is and remains disabled. When the data obtained from the identifier matches predefined requirement, demands or scenarios of use the electronic unit may at least temporarily deactivate the at least one interlock and the drive mechanism becomes unlocked for providing at least one of setting and injecting the dose of the medicament.

When processing of the data is conducted by the at least one processor of the electronic unit of the injection device the NFC interface does not have to provide data processing or data pre-processing. The at least one processor may read or obtain the data or information from the identifier and unalterably transfer or transmit the respective information to the at least one processor of the electronic unit.

According to some embodiments, the housing of the injection device includes a longitudinal structure that extends along a longitudinal direction. The housing may include a distal housing component at a proximal housing component. The distal housing component, also denoted as cartridge holder, is configured to accommodate the medicament container. The proximal housing component is configured to accommodate or to support the drive mechanism. The distal housing component includes a distal end facing away the proximal housing component. The distal end of the distal housing component may be provided or connected with an injection needle so as to pierce or to puncture biological tissue for injecting the medicament.

The proximal end of the distal housing component is detachably connectable to a distal end of the proximal housing component. By detaching or disconnecting the distal housing component from the proximal housing component the medicament container, e.g., a cartridge filled with the medicament, can be replaced on demand.

Moreover, the drive mechanism provided on or in the proximal housing component is resettable. When the housing components are disconnected the drive mechanism may be subject to a reset operation so that the injection device can be set into an initial configuration after inserting a new medicament container into the distal housing component and upon reconnecting the distal housing component with the proximal housing component.

According to some embodiments, the identifier is located on or inside the distal housing component. The electronic unit is provided in or on the proximal housing component. The electronic unit and/or the at least one interlock is mechanically engaged with at least one movable component of the drive mechanism. The electronic unit may be provided inside the proximal housing component or arranged at or near a proximal end of the proximal housing component. With the identifier located on or inside the distal housing component the identifier may be located rather remote from the electronic unit and/or remote from the NFC interface of the electronic unit.

In some embodiments, the identifier is located on the medicament container. In some embodiments, the identifier is located on the distal housing component, where the distal housing component is preassembled with the medicament container. The medicament container may be non-detachably connected to the distal housing component. The distal housing component can be detachably connectable and/or detachably fixable to the proximal housing component.

According to some embodiments, the identifier includes at least one of an optical code and a visible code. The optical code or visible code can be captured by an optical sensor of the electronic unit. Alternatively, there may be provided an external electronic device including an optical sensor operable to capture or to read the optical or a visible code.

With either implementation the optical sensor may be equipped with a two-dimensional sensor array. The optical sensor may include an imaging sensor so as to capture a two- or one-dimensional image of the optical code. In some embodiments, the electronic unit includes a camera to capture an image of the optical or visual code. In some embodiments, the external electronic device includes a camera to capture an image of the optical or visual code. The external electronic device may include a NFC interface to communicate with the NFC interface of the electronic unit of the injection device. With further examples, the external electronic device includes a short-range communication interface operable to communicate with a corresponding short-range communication interface of the injection device.

The external electronic device may be further configured to extract code information or medicament related information from the captured optical or visible code and to provide or to transmit the extracted data or information to the electronic unit via a wireless communication link between the short-range communication interface or the NFC interface of the electronic unit and the corresponding short-range or communication interface or NFC interface of the external electronic device.

With the external electronic device, it is generally possible to assemble the distal and proximal housing component with the medicament container located inside the housing of the injection device and to subsequently conduct a validation or approval routine of the medicament container by the identifier, the external electronic device and any of the near-field or short-range communication between the external electronic device and the electronic unit so as to at least temporarily deactivate the interlock of the injection device.

In some embodiments, the identifier includes a passive electronic identifier. A passive electronic identifier may electronically communicate either directly with the electronic unit of the injection device or with the external electronic device. The passive electronic identifier may be incompatible with an optional short-range communication interface of the electronic unit of the injection device. Such an incompatibility can be bridged or bypassed by the external electronic device operable to read the identifier and operable to communicate with any of the NFC interface and the short-range communication interface of the electronic unit of the injection device.

In some embodiments, the identifier is read by way of the NFC interface of the electronic unit of the injection device. To do so, the identifier is brought in close proximity to the NFC interface, e.g., before assembling the distal housing component to the proximal housing component or before arranging the medicament container provided with the identifier inside the distal housing component. When the medicament container is assembled inside the hosing of the injection device a spatial distance between the NFC interface, e.g., provided at or near the proximal end of the housing, and the identifier is larger than a transmission range of the near-field transmission interface.

According to some embodiments, the passive electronic identifier is an unpowered electronic identifier being void of a permanent source of electric energy. This allows for reducing manufacturing costs and saving storage space for implementing the identifier on the medicament container or on the distal housing component. A passive or unpowered electronic identifier can be manufactured in large quantities at low or moderate costs, and can be implemented or configured as a disposable electronic identifier.

According to some embodiments, the passive electronic identifier includes a microchip and an antenna. The antenna is configured to harvest electric energy from a surrounding electromagnetic field. In some embodiments, the passive electronic identifier is implemented as a NFC (NFC) tag. In some embodiments, the passive electronic identifier includes a passive RFID tag. Furthermore, and In some embodiments, the passive electronic identifier includes a NFC tag configured to communicate with a corresponding NFC interface of the electronic unit and/or of the external electronic device.

According to some embodiments, the electronic identifier includes a nonvolatile memory to store at least one of a medicament name, a medicament manufacturing date, a medicament expiry date, a LOT number of the medicament or a unique medicament container ID. In this way, a rather large variety of medicament related data can be stored in or by the identifier and can be captured or retrieved through a readout of the identifier.

The above-mentioned medicament-related parameters, such as medicament name, manufacturing date, expiry date, LOT number or an unique medicament container ID may be also encoded in an optical or visual code to be captured by the optical sensor, e.g., a camera. In some embodiments, the visible code may include a QR code or a data matrix code which can be translated into respective data via a network connection with a respective decoder.

According to some embodiments, the nonvolatile memory of the electronic identifier is at least one of erasable or writable by the NFC interface of the electronic unit. In this way, the nonvolatile memory of the passive electronic identifier can be reconfigured. There may be provided a validation flag in the nonvolatile memory indicating if the respective electronic identifier has been subject to a read or write operation before. By providing an erasable or writable nonvolatile memory, such a read or invalidation flag can be set or reconfigured upon a first reading or readout of the nonvolatile memory by the NFC interface of the electronic unit or of the external electronic device.

In this way, patient safety can be enhanced so as to prevent a multiple sequential use of one and the same medicament container, e.g., with the same or with different injection devices. In particular, a medicament container provided with a unique medicament container ID can be marked to be invalidated, so that an abuse of the container, e.g., a non-intended refilling of the container becomes somewhat effectless because then, such a counterfeited container will be inoperable to deactivate the interlock of the injection device.

In other embodiments, a transmission range of the NFC interface is smaller than a spatial distance between the NFC interface and the identifier when the medicament container is operably engaged with the drive mechanism. The medicament container can be arranged inside the distal housing component, which in turn is connected to the proximal housing component.

When the identifier is outside the transmission range of the NFC interface of the electronic unit and assembled inside the housing of the injection device, a reading or read-out procedure of the identifier is conducted before the medicament container is assembled inside the housing. For this, the medicament container and/or the distal housing component provided with the identifier is/are brought in close proximity to the electronic unit before a final assembly of the distal housing component and/or of the medicament container takes place. This way a read-out procedure as well as capturing of data or information provided by the identifier can be conducted when implementing the identifier as a passive electronic identifier with a limited transmission range.

In other embodiments, the electronic unit includes a NFC interface and a short-range communication interface to communicate with a corresponding short-range communication interface or NFC interface of an external electronic device.

With some embodiments, the electronic unit and/or the external electronic device may each include two communication interfaces, namely a NFC interface to communicate with the passive electronic identifier provided on or associated with the medicament container and a short-range communication interface, e.g., providing a communication link between the electronic unit and the external electronic device.

When the identifier is implemented as an NFC tag, e.g., a RFID tag, the identifier may be inoperable to communicate with a short-range communication interface of the electronic unit or the injection device. The external electronic device may be compatible with both, the NFC interface and the short-range communication interface of the electronic unit and may provide a relay function so as to capture or to obtain information from the electronic identifier and to transmit the respective information to the electronic unit, e.g., via the short-range communication interfaces.

The communication link between the electronic unit and hence the NFC interface thereof and the passive electronic identifier may be of bidirectional type. Hence, the electronic unit may not only be operable to read or to capture information from the passive electronic identifier but may be also operable to transmit data to the passive electronic identifier and/or to store or to modify a nonvolatile memory of the passive electronic identifier.

In this way, once the passive electronic identifier has been read out by the NFC interface of the electronic unit or of the external electronic device, the passive electronic identifier of the injection device may be invalidated. The passive electronic identifier may be electronically marked as being in use or that the associated medicament container has been used and cannot be used any further, e.g., with another injection device.

In other embodiments, the transmission range of the NFC interface is smaller than a transmission range of the short-range communication interface. The short-range communication interface may provide unidirectional or bidirectional communication between the electronic unit and the external electronic device. The NFC interface may be particularly configured for reading of the identifier provided on or associated with the medicament container. The NFC interface and the short-range communication interface may communicate with each other, e.g., by a processor of the electronic circuit. This way, signals or information received by one of the short-range communication interface and the NFC interface can be provided or transmitted to the other one of the short-range communication interface and the NFC interface.

In other embodiments, the short-range communication interface of the electronic unit or of the external electronic device includes a wireless communication interface based on one of the following communication standards, Bluetooth, Bluetooth low energy (BLE), ZigBee or Wi-Fi. The short-range communication interface of the electronic unit and the corresponding short-range communication interface of the external electronic device includes a transmission range that is larger than a transmission range of the NFC between the NFC tag of the identifier and the NFC interface of the electronic unit or external electronic device.

With some embodiments, the spatial distance between the identifier provided on the medicament container or the distal housing component and the electronic unit of the injection device is larger than the transmission range of the NFC interface. To read the identifier, the identifier, e.g., the medicament container or the distal housing component, is brought in close proximity to the electronic unit to establish and enable an NFC link between the identifier and the NFC interface of the electronic unit.

In some embodiments, the external electronic device (e.g., a portable electronic device) provides a relay function to enable indirect communication between the electronic identifier provided on or associated with the medicament container and the electronic unit, e.g., provided at the proximal end of the housing of the injection device.

According to some embodiments, the at least one interlock of the drive mechanism includes an electromechanical interlock configured to mechanically engage with a first component of the injection device and with a second component of the injection device. The first component is movable relative to the second component for at least one of setting of the dose and injecting of the dose of the medicament. The at least one interlock is operable to switch between an interlocked state, in which the first component is movably locked to the second component and a release state, in which the first component is movable relative to the second component, for at least one of setting of the dose and dispensing or injecting of the dose.

In some embodiments, the first component is movable in longitudinal direction relative to the second component for at least one of setting of the dose and injecting of the dose. In some embodiments, the first component is rotatable relative to the second component for at least one of setting of the dose and injecting of the dose. With a rotatable relative displacement between the first component and the second component an axis of rotation can extend along the longitudinal direction or axial direction of the housing of the injection device.

In some embodiments, the first component is subject to a helical motion relative to the second component for setting and/or injecting of the dose of the medicament. At least one of the first and the second components may belong to the drive mechanism. With some embodiments, both, the first and the second components belong to the drive mechanism of the injection device. In some embodiments, at least one of the first and the second component is or forms part of the housing of the injection device.

In other embodiments, the electromechanical interlock includes an electrically implemented actuator operably engaged with a latch. In some embodiments, the latch is movable by the actuator along a longitudinal direction, along a radial direction and/or along a tangential direction with regards to a cylindrical or tubular geometry of the housing of the injection device. The actuator is located and/or fixed on one of the first and second components. The latch may be provided on the same component and may be mechanically engageable with the other one of the first and second component to block or to impede a relative movement of the first component and the second component when the interlock is in the interlocked state.

In some embodiments, the drive mechanism of the injection device includes a piston rod, a number sleeve, a drive sleeve or driver and at least one of a dose dial and a trigger or dose button. For setting of a dose, the dose dial may be rotatable relative to the housing. During dose setting, the rotational movement of the dial is transferred to a respective rotational movement of the number sleeve. For impeding setting of a dose, at least one of the dose dial and the number sleeve may be rotationally and/or translationally locked by the interlock.

For dispensing or injecting of the dose, the trigger or dose button is depressed by a user. The trigger button may be depressible relative to the dose dial, the housing, the number sleeve, or the piston rod. During dose dispensing, the driver or drive sleeve may be subject to a rotational movement that is transferred into a longitudinal and/or rotational movement of the piston rod. During dose injection, the number sleeve rotates in a direction opposite to a direction of rotation during setting of the dose. The interlock may be operable to block actuation or movement of the trigger, to block a rotation of the driver or drive sleeve, to block a rotation or movement of the piston rod and/or to block or to inhibit rotation or movement of the number sleeve.

Further implementations of the interlock or electromechanical interlock are possible and conceivable, as long as the first and second component to be mutually locked and engaged are subject to a relative movement during at least one of setting of the dose and dispensing of the dose.

According to some embodiments, the drive mechanism includes at least one clutch operable to switch the drive mechanism between a dose setting mode and a dose injecting or dose dispensing mode. The at least one interlock is operably engaged with the at least one clutch to selectively disable switching of the drive mechanism from one of the dose setting mode and the dose injecting mode into the other one of the dose setting mode and the dose injecting mode.

In some embodiments, the drive mechanism includes a first clutch and a second clutch. By way of a first clutch the number sleeve can be selectively engaged with a dose dial, a driver or drive sleeve of the drive mechanism. In this way, a rotation of the dose dial can be transferred into a respective rotation of the number sleeve during setting of the dose. During injecting of the dose the number sleeve can be decoupled and released from the dose dial. The number sleeve is allowed to rotate in an opposite sense of rotation while the dose dial does not have to follow this rotation but remains rotationally locked to the housing of the injection device.

By way of a second clutch the dose dial and/or the number sleeve can be selectively engaged with a driver or drive sleeve of the drive mechanism. Furthermore, by way of the second clutch and/or a third clutch, a selective engagement between a driver or drive sleeve and the piston rod can be provided to move the piston rod in distal direction during injecting of the dose.

Generally, the drive mechanism of the injection device can be implemented in various different ways. Some embodiments of a drive mechanism is for example disclosed in WO 2004/078239 A1, the entirety of which is incorporated herein by reference. Another example of a drive mechanism to be implemented with the injection device is for example disclosed in WO 2014/033195 A1, the entirety of which is incorporated herein by reference.

According to some embodiments, the at least one interlock is controlled by a processor of the electronic unit. The at least one processor is configured to activate the at least one interlock if one or several of the following conditions have been detected by the electronic unit. Such conditions may be: end of a dose injection, depletion of the medicament container, lapse of a predefined time interval since deactivation of the at least one interlock, removal of the medicament container from the housing, insertion of the medicament container into the housing, conducting of a manual reset of the drive mechanism or lapse of a predefined time interval since a communication link between the NFC interface of the electronic unit and the identifier abrogated.

There may be other conditions by way of which the at least one processor is triggered to activate or to de-activate the interlock. With any of the above conditions, an automated activation of the interlock can be provided, thereby effectively preventing unintended or non-authorized use of the injection device. Any of the above-mentioned conditions can be detected e.g., by at least one sensor as described further below,

According to some embodiments, the injection device includes the medicament container filled with the medicament. The medicament container is arranged inside the housing. The medicament container includes a cartridge filled with the medicament. The cartridge may include a tubular-shaped barrel, e.g., made of a vitreous material. The barrel may be sealed towards the proximal direction by a stopper movable along the longitudinal direction of the barrel for expelling the liquid medicament through a distally located outlet of the barrel.

The distally located outlet may be sealed by a seal. The seal at the distal end of the cartridge may include a sealing disc, e.g., made of a rubber material and being pierceable by a double-tipped injection needle. The rubber seal may be fixed or assembled to the distal end of the tubular barrel by way of a crimped metal cap.

According to some embodiments, the medicament container is non-removably attached to the distal housing component. The distal housing component includes a tubular-shaped cartridge holder shaped and configured to accommodate a medicament cartridge therein. The cartridge holder may be provided with a snap feature to fix the cartridge inside the cartridge holder. In this way, a cartridge holder cartridge pre-assembly may be commercially distributed to end consumers or patients. With such a preassembly, the identifier may be provided on an outside surface of the distal housing component, hence on an outside surface of the cartridge holder.

In some embodiments, the identifier is provided on an outside surface of the barrel of the medicament container. In some embodiments, the identifier is provided on a stopper of the cartridge or inside the stopper of the cartridge.

When the identifier is located on or in the medicament container, the identifier may be readable by the external electronic device, even when the cartridge or medicament container is located at/or assembled inside the distal housing component. For this, the distal housing component together with the medicament container assembled therein may be arranged in close proximity to the NFC interface of the electronic unit of the injection device. Additionally, and to facilitate readout of the identifier there may be provided a window or an aperture in a sidewall of the distal housing component.

Alternatively, or additionally, the sidewall of the distal housing component is transparent or translucent.

With some embodiments, e.g., when the identifier is implemented as a passive electronic identifier the sidewall of the distal housing component may be permeable for electromagnetic radiation by way of which the passive electronic identifier can be read out.

According to some embodiments, the electronic unit of the injection device includes at least one sensor to detect or to measure movement of at least one movable component of the injection device. The movable component of the injection device may be indicative of a particular device configuration or device operation. By detecting the movement or the magnitude of the movement of the movable component the electronic unit is provided with information regarding the operational status of the injection device.

In some embodiments, the sensor is operable to quantitatively measure the degree of movement of the at least one movable component. The at least one sensor may be operable to measure a degree of rotation and/or a degree of longitudinal movement of the at least one movable component, e.g., relative to the housing of the injection device or relative to another movable component of the drive mechanism of the injection device.

By way of the at least one sensor, data pertaining to dose size can be derived. The at least one sensor is operable to provide electrical signals being indicative of a size of a dose actually set and/or dispensed or injected by the drive mechanism of the injection device. The electronic unit is provided with a memory as well as with a clock. A dosing history or injection history can be automatically captured and stored in the memory of the electronic unit.

By way of the optional short-range communication interface and by way of a communication link with an external electronic device, dispensing-related data and hence injection-related data, such as an injection history can be synchronized with the external electronic device and transmitted to medical service providers for further data analysis. In this way, repeated use of the injection device can be precisely tracked and measured and the user's compliance with a given prescription schedule can be monitored.

According to some embodiments, the present disclosure relates to a kit of injection devices, in particular to an injection device of a first type and at least to an injection device of a further type, e.g., a second type. Injection devices of the first type and of the second type distinguish by their medicament or medicament container. The medicament container of the first type may distinguish from a medicament container of the second type by size, the type of medicament, the pharmaceutically active substance and/or by the concentration of the pharmaceutically active substance.

The medicament container of the first type is configured for use with a drive mechanism of a first type. The medicament container of the second type is configured for use with a drive mechanism of a second type. Unintended cross use, e.g., use of a medicament container of the first type with an injection mechanism of the second type should be avoided.

For this, the medicament container of the first type is equipped and/or associated with an identifier of the first type. The medicament container of the second type is equipped and/or associated with an identifier of the second type. By way of the identifier, the respective type of the medicament container can be detected. Here, the identifier provides a kind of a coding. The identifier of the first type represents a first coding. The identifier of the second type represents a second coding. The identifiers of different types are processable and/or detectable by the electronic unit so as to activate or to deactivate the interlock.

The identifiers may represent an electronic coding or a coding that is electronically detectable. Such identifiers or codings enable use of mechanically unencoded housing components for the injection devices of the first and the second type. Even though a distal housing component equipped with a medicament container of the first type is assembled with a proximal housing component of a second type and equipped with a second type of an injection device, such a combination may not be operable to abrogate or to deactivate the interlock of the drive mechanism. Setting and/or injecting of a dose will be effectively impeded.

According to some embodiments, the present disclosure also relates to an electronic device. The electronic device includes a short-range communication interface operable to communicate with a short-range communication interface of an injection device as described above. Optionally, the electronic device further includes a wireless reader interface directly or indirectly coupled to the short-range communication interface and operable to capture or to read the identifier provided on or associated with the medicament container of the injection device.

The electronic device may be implemented as a mobile or wearable electronic device, such as a smart phone, a smart watch or a tablet computer. The electronic device may provide a relay for reading or capturing information of the identifier provided on or associated with the medicament container and being operable to transmit or forward this information to the short-range communication interface of the electronic unit of the injection device. The electronic device may unalterably transfer or transmit the information to the electronic unit of the injection device as gathered from identifier.

Generally, the electronic device is configured to communicate and/or to cooperate with the injection device as described above. Insofar, all features, effects and benefits as described above in connection with the injection device equally apply to the electronic device.

According to some embodiments, the electronic device, in particular the wireless reader interface thereof includes at least one of a camera to capture an optical code of the identifier and a NFC interface to communicate with a passive electronic identifier, e.g., with a NFC tag of the identifier. In either way, the wireless reader interface of the external electronic device is operable to acquire, to capture, to read or to retrieve information of the identifier and to forward this information to the electronic unit of the injection device via the short-range communication link between the short-range communication interface of the electronic unit of the injection device and a corresponding short-range communication interface of the electronic device.

According to some embodiments, the electronic device includes a wide range communication interface configured to communicate with a database via a network. The wide range communication interface may be implemented as a mobile phone-based communication interface, such as USM, LTE, 4G, 5G, 6G or any other telecommunication standards. The wide range communication interface may be implemented to communicate in a wireless wide area network (WANW). Such networks are created through the use of mobile phone signals, which are provided and maintained by specific mobile phone (cellular) service providers.

The short-range communication interface may be implemented as a wireless local area network (WLAN). Such networks are wireless networks that use radio waves. The backbone network usually uses cables, with one or more wireless access points connecting the wireless users to the wired network. The transmission range of such WLAN network can be anywhere from a single room to an entire campus. The short-range communication interface may be also implemented to communicate with a wireless personal area network (WPAN), which can be implemented as a short-range network that uses Bluetooth or Bluetooth low energy (BLE). The short-range communication interface may provide a transmission range of several meters.

The NFC interface as described above uses a communication protocol for communication between two respective electronic devices over a distance of only a few centimeters or less. The transmission range of the NFC interface is less than or equal to 4 cm, less than or equal to 3 cm, less than or equal to 2 cm. The NFC interface may be based on inductive coupling between two so-called antennas present on NFC-enabled devices communicating in one or both directions, using a frequency of 13.56 MHz in the globally available unlicensed radio frequency ISM band using the ISO/IEC 18000-3 air interface standard at data rates ranging from 106 to 424 kbit/s.

With the electronic device including a wide range communication interface and/or by way of the short-range communication interface the electronic device and/or the electronic unit of the injection device is configured to connect to a database via a network, such as the internet. In this way, communication with a database is enabled and data synchronization is provided. Data from the storage and/or data provided by the database can be compared and evaluated with data obtained from the identifier.

The electronic device and/or the electronic unit may be implemented to transmit identifier related data to the database and to retrieve respective data from the database in return. This way, information about the identifier and hence information about the medicament container can be synchronized with the data provided in the database. Data evaluation or data approval can be performed. For example, and by way of the communication with the database a checkup can be conducted e.g., if a unique identifier associated with the medicament container is a valid identifier or if such an identifier has been marked as invalid in the database.

In this way, each individual medicament container can be tracked and at least virtually marked in the database as being valid or invalid. A valid identifier as stored in the external database can be marked or over written as invalid once the identifier has been detected by the electronic unit or external electronic device and through data communication with the database via the network. This way, counterfeited containers can be precisely detected and patient safety can be thus improved. In effect, only original or genuine medicament containers provided with a valid identifier can be used to unlock the drive mechanism of the injection device.

According to some embodiments, the present disclosure also relates to an injection system. The injection system includes an injection device as described above, optionally equipped with a medicament container. The injection system further includes an electronic device as described above. Insofar, any features, effects and benefits as described above in connection with the injection device and/or in connection with the electronic device equally apply to the injection system; and vice versa.

According to some embodiments, the present disclosure relates to a method of unlocking operation of an injection device for at least one of setting or injecting of a dose of a medicament. The injection device includes a housing configured to accommodate a medicament container and a drive mechanism. The drive mechanism is operably engageable with the medicament container for expelling or withdrawing the dose of the medicament from the medicament container. The drive mechanism includes a piston rod to exert a distally directed pressure onto a stopper movably arranged in a barrel of the medicament container.

The drive mechanism further includes at least one interlock configured to disable at least one of setting of the dose and dispensing of the dose. The method of unlocking operation of the injection device includes the step of capturing or reading of an identifier provided on or associated with the medicament container. In some embodiments, the identifier is provided on the medicament container or is integrated into the medicament container. In some embodiments, the medicament container may be preassembled in a distal housing component of the injection device. The identifier may be attached to the distal housing component or integrated into the distal housing component.

The method includes the step of capturing or reading of the identifier provided on or associated with the medicament container by a NFC interface of an electronic unit of the injection device.

Furthermore, the identifier is validated or approved by comparing medicament-related data obtained from the identifier with predefined data related to the injection device.

The method further includes the step of at least temporarily deactivating or re-activating the at least one interlock on the basis of the validation or approval of the identifier.

Generally, the method of unlocking operation of the injection device is implemented by an injection device as described above. Insofar, all features, effects and benefits described above in connection with the injection device, the electronic device and the injection system equally apply to the method of unlocking operation of the injection device; and vice versa.

According to some embodiments, the method includes the step of validating the identifier by comparing medicament-related data obtained from the identifier with predefined data related to the injection device. Validating the identifier may include determining/asserting if the identifier matches with predefined data related to the injection device and if the medicament container is usable or is intended for use with the injection device.

If the acquired or captured identifier-related data does not match with predefined data related to the injection device, the drive mechanism is either actively locked or remains locked by the interlock. As such, the injection device is effectively inoperable to set a dose of the medicament and/or to inject a dose of the medicament. In some embodiments, validating the identifier may include modifying the identifier. This may particularly apply through the NFC link between the electronic identifier and the NFC interface of the electronic unit of the injection device.

In some embodiments, the electronic unit of the injection device may be operable to read out the electronic storage of the passive electronic identifier. Upon such a readout activity a respective readout flag of the electronic identifier may be set so as to mark the electronic identifier as being currently in use or as having been used. A repeated use of the passive electronic identifier and/or of the medicament container associated with the identifier may be then effectively impeded or blocked, e.g., to prevent or to impede an unintended and/or unauthorized use of the medicament container. Insofar, capturing or reading of the identifier may include an evaluation or approval of the status of the readout flag provided by the electronic storage of the electronic identifier.

Alternatively, upon readout of the identifier provided on or associated with the medicament container by the NFC interface, a database request is invoked with an external database, e.g., via a network connection. In the database, the medicament container currently in use may be marked as ready for use or unsuitable for use. With such a database request additional information may be gathered if a particular medicament container can be used or should not be used or cannot be used with the injection device.

Accordingly, an NFC link between the electronic unit of the injection device and the identifier may be used to control, e.g., to lock or to release the interlock so as to enable or to disable operation of the injection device with the given medicament container.

According to some embodiments, capturing or reading of the identifier includes the step of at least temporarily arranging or bringing the medicament container within a maximum distance to the NFC interface. The maximum distance is smaller than or equal to the transmission range of the NFC interface. If the electronic unit is arranged at or near the proximal end of the housing of the injection device, the identifier, e.g., the respective medicament container or distal housing component, is brought in close proximity to the proximal end of the housing of the injection device (e.g., to a proximal end of the proximal housing component), thereby arranging the identifier within the transmission range of the NFC interface to enable a readout of the identifier and/or to support capturing/acquiring respective identifier data or identifier information.

According to some embodiments, the present disclosure relates to a computer-readable medium including instructions which, when executed by an electronic unit of an injection device as described above causes the electronic unit to execute the steps of capturing or reading of an identifier provided on or being associated with the medicament container, validating the identifier by comparing medicament-related data obtained from the identifier with predefined data related to the injection device and to at least temporarily deactivate or re-activate the at least one interlock on the basis of the validation of the identifier.

The computer-readable medium is configured to execute the steps of the above-mentioned method of unlocking operation of the injection device. Insofar, all features, effects and benefits as described above with the method of unlocking also apply to the computer-readable medium, mutatis mutandis.

According to some embodiments, the computer-readable medium may include instructions which, when executed by the at least one processor of the electronic unit of the injection device cause the at least one processor to control the interlock of the drive mechanism of the injection device. The computer-readable medium may include instructions, which, when executed by the at least one processor of the electronic unit cause the electronic unit to process data obtained from the identifier and to lock or to unlock the at least one interlock on the basis of the captured data or signals derived from the identifier. The computer-readable medium is particularly executable by the electronic unit of the injection device. Insofar, all features, effects and benefits as described above in connection with the injection device equally apply to the respective computer-readable medium; and vice versa.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context, the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein, and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., the antibody has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted that any reference numerals used in the appended claims are not to be construed as limiting the scope of the disclosure.

### BRIEF DESCRIPTION OF FIGURES

FIG. 1 schematically illustrates a longitudinal cross-section of some embodiments of an injection device.

FIG. 2 schematically shows the injection device according to FIG. 1, with the distal housing component disconnected from the proximal housing component.

FIG. 3 shows a longitudinal cross-section through an example of a drive mechanism implemented with the injection device.

FIG. 4 shows the interaction between the electronic unit of the injection device with an external electronic device.

FIG. 5a is a block diagram of the injection system including an injection device and an external electronic device.

FIG. 5b is a block diagram of a configuration for reading of an identifier.

FIG. 6 shows a distal housing component provided with an identifier.

FIG. 7 shows a distal housing component and a medicament container provided with the identifier.

FIG. 8 shows an example of an electronic identifier.

FIG. 9 shows an example of an optically implemented identifier.

FIG. 10 shows a flowchart of a method of unlocking operation of the injection device.

### DETAILED DESCRIPTION

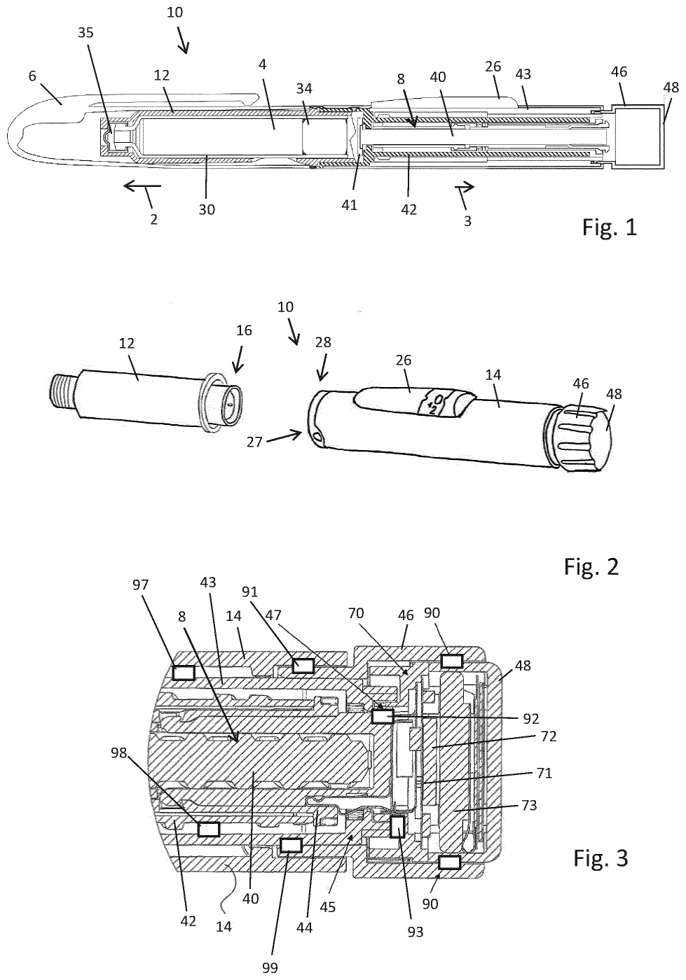

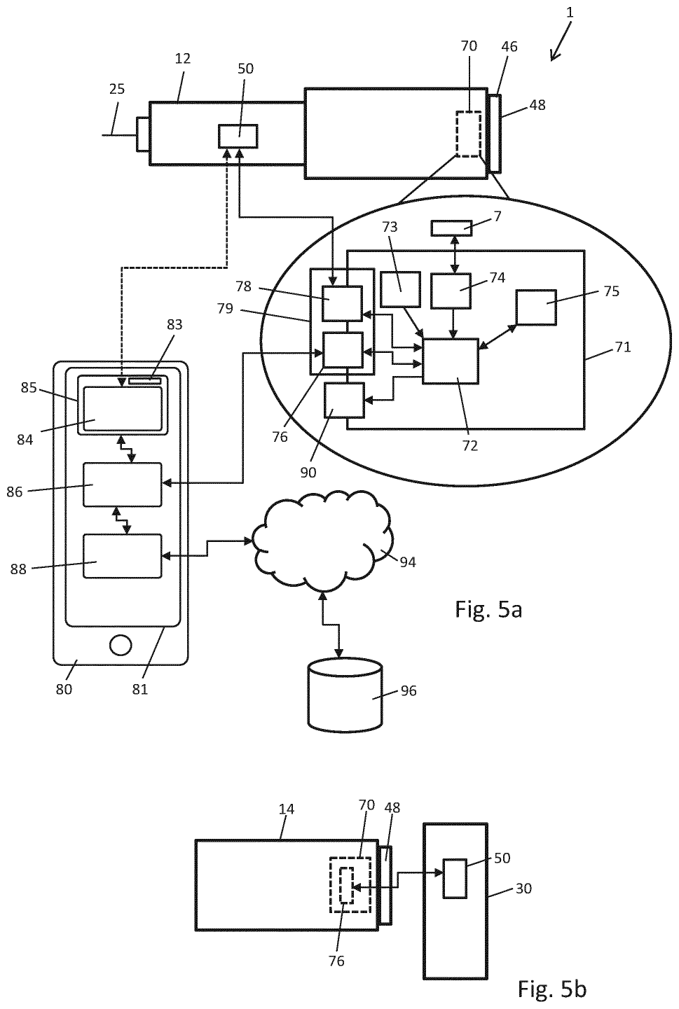

In FIGS. 1-3, 6 and 7 details of an example of an injection device 10 implemented as a pen-type injector are schematically illustrated. The injection device 10 is part of an injection system or drug delivery system 1 as illustrated in FIG. 5. The injection device 10 includes a housing 11. The housing 11 may be of elongated or tubular shape. The housing 11 extends in a longitudinal or axial direction. Towards the distal direction 2 the injection device 10 includes a protective cap 6 covering a distal housing component 12. The housing 11 further includes a proximal housing component 14 detachably connectable with the distal housing component 12. As becomes apparent from FIGS. 1, 2 and 6 the proximal housing component 14 includes a connecting end towards the distal direction 2.

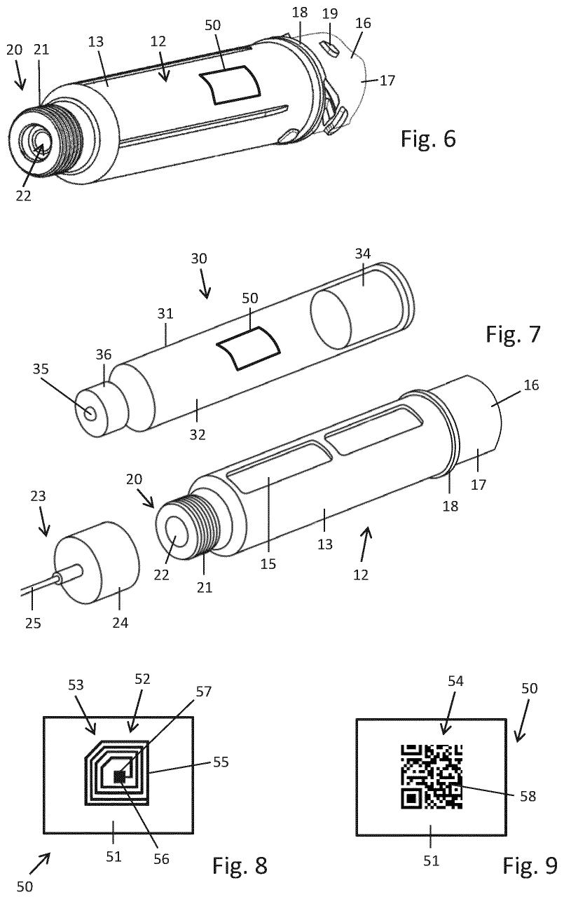

The connecting end 28 of the proximal housing component 14 includes a receptacle 27 configured to receive a complementary shaped proximal connecting end 16 of the distal housing component 12. As illustrated in FIGS. 2 and 6, the proximal connecting end 16 of the distal housing component 12 includes an insert portion 17 configured for connection with a distal connecting end 28 of the proximal housing component 14. For this, the proximal housing component 14 includes a receptacle 27 at the distal connecting end 28 configured and shaped to receive the insert portion 17 therein.

On an outside surface of the insert portion 17 there is provided at least one fastening element 19, e.g., implemented as a protrusion to mate or to engage with a complementary-shaped recess on an inside surface of a sidewall of the receptacle 27. The insert portion 17 and the receptacle 27 may include mutually corresponding or complementary shaped fastening elements, so as to form a snap fit engagement, a bayonet joint or a threaded joint.

The insert portion 17 is delimited in longitudinal direction by a radially outwardly extending flange 18. When the housing components are fully assembled, the flange 18 is in proximal longitudinal abutment with a distal end face of a sidewall or of a distal end of the proximal housing component 14.

The distal housing component 12 is implemented as a cartridge holder, and includes a tubular-shaped sidewall 13 configured and sized to receive a medicament container 30 as illustrated in FIG. 7. The cartridge holder as shown in FIG. 7 includes at least one aperture 15 in the tubular-shaped sidewall 13. Towards the distal end the tubular-shaped sidewall 13 radially narrows and forms a shoulder portion adjoining into a distal connecting end 20. The medicament container 30 can be filled with an injectable medicament 4, e.g., provided or stored in liquid form inside the medicament container 30.

The connecting end 20 includes a mount 21 for a needle assembly 23. The needle assembly 23 includes a needle hub 24 configured for detachable or releasable engagement with the mount 21 and further including an injection needle 25. The injection needle 25 can be implemented as a double-tipped injection needle. When correctly assembled on the mount 21 a proximal tipped end of the injection needle 25 reaches through a distal through opening 22 at the connecting and 20 and pierces a seal 35 provided at the distal end of the medicament container 30 located inside the cartridge holder.

The seal 35 may be implemented as a rubber disc, and may serve as a pierceable septum. The seal 35 may be mechanically fixed to the distal end of the barrel 31 of the medicament container 30 by way of a crimped metal cap 36. Towards the proximal end the tubular shaped barrel 31 of the medicament container 30 is sealed by a stopper 34, e.g., implemented as a rubber stopper. The piston or stopper 34 is movably disposed inside the barrel 31, and may be displaceable towards the distal direction 2, hence towards the dispensing end of the injection device 10 through the action of a piston rod 40 of a drive mechanism 8 provided and assembled inside the proximal housing component 14.

The drive mechanism 8 can be implemented in many different ways. Insofar the present disclosure is by no way limiting to the presently illustrated example of a particular drive mechanism 8. In the presently illustrated example, the drive mechanism 8 includes a piston rod 40 equipped with a pressure piece 41 at the distal end. The pressure piece 41 is configured to apply distally directed pressure onto the stopper 34 of the medicament container 30. The drive mechanism or the injection device further includes a housing insert 42, which serves as a mount for numerous movable components of the drive mechanism 8.

The drive mechanism 8 includes a number sleeve 43 provided with a consecutive sequence of numbers on an outside facing sidewall. These numbers are visible through a window 26 or through a respective aperture in the sidewall of the proximal housing component 14. The drive mechanism 8 further includes a driver 44, which may be implemented as a drive sleeve. For setting of a dose, the number sleeve 43 may be subject to a rotation relative to the housing 11 along a first sense of rotation. During dispensing or injecting of the dose the number sleeve 43 is subject to a rotation along a second and hence opposite sense of rotation.

The driver 44 or drive sleeve can be permanently or non-permanently operably engaged with the piston rod 40. At least during dispensing of a dose the driver 44 is in a force-or torque-transmitting engagement with the piston rod so as to urge the piston rod 40 in distal direction 2 for dose expelling or dose injecting. With some implementations of the drive mechanism 8, the driver 44 is subject to a rotation along the second sense of rotation during dose dispensing. In some embodiments, the driver 44 is subject to a rotation along the first sense of rotation during and/or for setting of the dose. In some embodiments, the driver 44 is rotationally and/or axially fixed relative to the housing 11 during setting of the dose but is movable during dispensing or injecting of the dose.

The drive mechanism 8 further includes a dose dial 46, e.g., provided at the proximal end of the proximal housing component 14. The dose dial 46 provides a user grippable or user operable tool for setting of the dose. The dose dial 46 is rotatable by a user relative to the housing 11 for setting of the dose. The drive mechanism 8 further includes a trigger 48, e.g., implemented as a dose button depressible by a user in distal direction 2 for initiating and/or for controlling the process of dose dispensing or dose injection.

The trigger 48 and the dose dial 46 may be provided on a common part, and may be integrally formed. The trigger 48 and the dose dial 46 may be both located on a so-called dial extension of the injection device 10, which is subject to a helical motion relative to the housing 11 during setting of the dose and which is subject to a longitudinal distally directed movement during and/or for dispensing or injecting of the dose. The trigger 48 is provided at a proximal end of the dose dial 46, and may form or constitute a proximal end face of the housing 11.

In the example of FIG. 3, numerous possible implementations of an interlock 90, 91, 92, 93, 97, 98, 99 are shown. The interlock 90 is provided as an interlock between the trigger 48 and the dose dial 46. With an activated interlock 90, a mutual displacement of the trigger 48 relative to the dose dial 46 can be effectively blocked. When the trigger 48 should be non-rotationally fastened to the housing 11 or to the dial extension an activated interlock 90 may also block a rotation of the dose dial 46 relative to the trigger 48 and hence relative to the housing 11. In this way, dispensing of a dose and/or dialing or setting of a dose can be effectively blocked or deactivated by activation of the interlock 90.

The interlock 91 is in mechanical connection with the proximal housing component 14 and with at least one of the dose dial 46, the trigger 48 and the dial extension. Activation of the interlock 91 serves to block or to impede at least one of a rotation and a longitudinal sliding movement of the dose dial 46 relative to the housing component 14. With an activated interlock 91 setting of a dose and/or dispensing of the dose can be effectively blocked.

The drive mechanism 8 may further include at least a first clutch 45 and a second clutch 47. By way of the clutches 45, 47 the drive mechanism 8 is switchable between a dose setting mode and a dose injecting mode. Depending on the specific implementation of the drive mechanism 8, the clutches 45, 47 are operable to mutually engage any two of the following components: piston rod 40, housing insert 42, housing 11, number sleeve 43, driver 44, dose dial 46 and/or trigger 48. Details of some embodiments of a drive mechanism are further described in Patent Publication No. WO 2014/033195 A1. The clutch 45 may be provided with a further interlock 93 and the clutch 47 may be provided with the interlock 92. The interlock 92 may be operably engaged with the clutch 47. Likewise, the interlock 93 may be operably engaged with the clutch 45.

With an activated interlock 92, 93 operation of the respective clutch 45, 47 may be blocked so as to impede a switching of the drive mechanism 8 from the dose setting mode into the dose injecting mode and/or vice versa, namely from the dose injecting mode into the dose setting mode.

As illustrated in FIG. 3, the number sleeve 43 is selectively engageable with the proximal housing component 14 via an interlock 97. In other embodiments, the number sleeve 43 is selectively engageable with the housing insert 42 via an interlock 98. In other embodiments, as illustrated in FIG. 3 the number sleeve 43 is selectively engageable with at least one of the dose dial 46 and the trigger 48 via another interlock 99.

By way of the interlock 97, at least one of a rotation and a longitudinal movement of the number sleeve 43 relative to the proximal housing component 14 can be blocked or impeded. By way of the interlock 98, at least one of a rotation and a longitudinal movement of the number sleeve 43 relative to the housing insert 42 can be selectively blocked or impeded. Since the number sleeve 43 is subject to at least one of a rotational, helical or longitudinal sliding movement relative to the proximal housing component 14 during setting of the dose and injecting of the dose, a respective blocking of a movement of the number sleeve 43 serves to block or to impede at least one of a dose setting and a dose injecting action.

By way of the interlock 99, a movement, e.g., a rotational and/or longitudinal movement of the dose dial 46 and/or of the trigger 48 can be blocked relative to the number sleeve 43. This way, injecting of a dose can be effectively impeded or prevented when the interlock 99 is activated.

In some implementations, the interlocks 90, 91, 92, 93,97, 98, 99 each include an electromechanical actuator and at least one of a latch and a clamp. This way, the latch or clamp is movable between a locking configuration and a release configuration by the actuator, where operation of the actuator is controllable by electrical signals, e.g., obtained from or generated by a processor 72 the function of which will be described below. The latch provided on a first component of the injection device 10 is configured to engage with a second component of the injection device in a form fitting manner.

In some embodiments, when the interlock includes a movable clamp, the clamp provided on a first component of the injection device is configured to engage with a second component of the injection device in a friction fitting manner. In either way and by activation of the interlock 90, 91, 92, 93, 97, 98, 99 there can be either provided at least one of a form fitting engagement and a frictional engagement between the first and the second component of the injection device so as to block or to impede at least one of setting of a dose and dispensing of a dose.

The actuator may include at least one of an electromagnet and an electromechanical drive controllable by the at least one processor 72. By way of the actuator the latch, e.g., implemented as a movable or elastically deformable bending arm, hook or clamp is movable between a locking position, in which the interlock 90, 91, 92, 93, 97, 98, 99 is activated, and a release position, in which the interlock 90, 91, 92, 93, 97, 98, 99 is deactivated. In the deactivated configuration the interlock 90, and 91, 92, 93, 97, 98, 99 allows and supports a movement of a first component relative to a second component of the drive mechanism so as to enable at least one of setting of a dose and injecting of the dose.

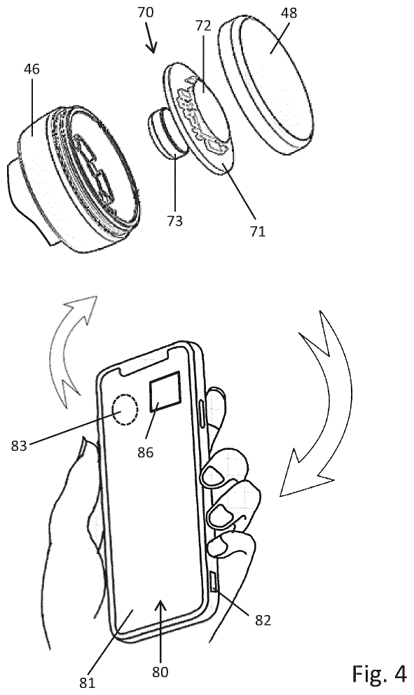

The injection device 10 is further provided with an electronic unit 70. In the present example, the electronic unit 70 is embedded inside the injection device 10, and is arranged at or near the proximal end of the housing 11. The electronic unit 70 may be located or arranged inside a dial extension of the injection device 10. In the example illustrated in FIGS. 3 and 4, the electronic unit 70 is arranged inside a hollow space of the cylindrically shaped dose dial 46, which is covered and/or closed towards the proximal direction 3 by the trigger 48. The trigger 48 provides an end cap forming a proximal closure of the hollow dose dial 46. In some embodiments, the trigger 48 and the dose dial 46 are integrally formed.

The electronic unit 70 includes a printed circuit board 71 provided with a processor 72. The electronic unit 70 is further provided with a source of electric energy, such as an electric battery 73. As illustrated, the at least one processor 72 and the battery 73 may be provided on opposite sides of the printed circuit board 71.

The electronic unit 70 is schematically illustrated in FIGS. 5a and 5b. The electronic unit 70 further includes a communication unit 79. The communication unit 79 includes a short-range communication interface 76, particularly configured to communicate with a corresponding short-range communication interface 86 of an external electronic device 80. The communication unit 79 further includes a NFC interface 78 configured to capture information of an identifier 50 provided on or associated with the medicament container 30. With the presently illustrated example the short-range communication interface 76 is optional whereas the NFC interface 78 is primarily used to obtain data from the identifier 50 to control unlocking of the interlock 90.

The at least one processor 72 is connected to the interlock 90 and is operable to control operation of the interlock 90. The interlock 90 may be implemented as an electromechanical interlock that includes a latch or any other type of a mechanically movable component to block or to impede operation of the drive mechanism 8.

Typically, the latch or the movable component of the interlock 90 is movable through and by an actuator that is electronically controllable by the at least one processor 72. By way of the short-range communication interface 76 the electronic unit 70 is operable to communicate with the external electronic device 80. Optionally, the electronic unit 70 is provided with a sensor 74 operable to detect or to measure movement of at least one movable component 7 of the drive mechanism 8. The movable component 7 may be represented by any of the above-mentioned movable components, e.g., the piston rod 40, the number sleeve 43, the driver 44, the trigger 48, the dose dial 46 and/or any of the clutches 45, 47. By way of the sensor 74, which may be implemented as one of an optical sensor, a capacitive sensor, an inductive sensor, an optical sensor or as an acoustic sensor, operation of the drive mechanism 8 and hence operation of the injection device 10 can be supervised and monitored.

By way of the sensor 74, information regarding the size of the dose currently set, dialed or dispensed can be obtained and can be stored in a local storage 75. Data repeatedly captured or obtained during repeated and subsequent use of the injection device 10 can be stored in the storage 75. Such injection-related data stored in the storage 75 can be synchronized by the short-range communication interface 76 with the external electronic device 80 and/or with an external database 96, e.g., hosted or provided by a healthcare provider.

The NFC interface 78 is implemented as an NFC reader operable to capture information from the passive electronic identifier 52, as illustrated in FIG. 8. The transmission range of the NFC interface 78 may be a few centimeters. With some embodiments, the spatial distance between the electronic unit 70, e.g., provided at or near the proximal end of the housing 11 and the identifier 50 is larger than a transmission range of the NFC interface 78. In some embodiments, the identifier 50 is placed or arranged in close proximity to the proximal end of the proximal housing component 14, as illustrated in FIG. 5b. The identifier 50 may be presented to the electronic unit 70 before the respective medicament container 30 and/or the distal housing component 12 readily equipped with the medicament container 30 is connected to the distal end of the proximal housing component 14.

In some embodiments, the optional external electronic device 80 includes a wireless reader interface 85 configured to read or to capture the identifier 50 provided on or associated with the medicament container 30. By way of the external electronic device 80, in particular by way of the wireless reader interface 85 an information content or data of the identifier 50 can be detected or read out by the external electronic device 80. Information gathered by the wireless reader interface 85 can be transmitted to the electronic unit 70 of the injection device via a communication link between the short-range communication interface 76 of the electronic unit 70 and the corresponding short-range communication interface 86 of the external electronic device 80.

The external electronic device 80 may be implemented as a smart phone, as a smart watch or as a tablet computer, and may include a wide range communication interface 88 to establish a communication link to the external database 96 by a communication network 94. The external electronic device 80 may be a mobile device or wearable device that includes a display 81 and/or a speaker 82 to communicate with a user.

The wireless reader interface 85 may also include a NFC interface 84 and/or a camera 83. The camera 83 may be operable to read the identifier 50 when implemented as an optical identifier 54 as illustrated in FIG. 9. The optical identifier 54 may include a one or two-dimensional optical code 58, e.g., a QR code or data matrix code capturable by the camera 83 of the external electronic device 80.

In the example illustrated in FIG. 8, the identifier 50 is implemented as a passive electronic identifier 52. The identifier 50 may include a NFC tag 53 provided with an antenna 55 and with a microchip 56. The microchip 56 may include a local storage or memory 57, e.g., to store medicament related information and/or or to store medicament container related information. The memory 57 can be implemented as a read-only memory or a re-writable memory.

In either way, the identifier 50 may include a planar substrate 51 for attaching the identifier 52 a sidewall 13 of the distal housing component 12 or to a sidewall of the barrel 31 of the medicament container 30. The planar substrate 51 may include a pliable or deformable sheet structure with an adhesive layer to adhesively attached the substrate 51 and hence the identifier 50 to the housing component 12 or to the medicament container 30.

The identifier 50, implemented as optical identifier 54 or as passive electronic identifier 52, can be non-detachably provided on or in the medicament container 30 and/or on or in the distal housing component 12. The information or data stored or provided by the identifier 50 may be indicative of a type or name of the medicament, a medicament manufacturing date, a medicament expiry date, a LOT number of the medicament and/or of a unique medicament container ID. The data or information provided by the identifier 50 can be acquired, retrieved, captured or obtained via the wireless reader interface 85 of the external electronic device 80.

The respective information can be either processed or pre-processed by the external electronic device 80 or can be simply forwarded or transmitted to the electronic unit 70 by the communication link between the short-range communication interface 76 and the corresponding short-range communication interface 86.

If the data of the identifier 50 is directly captured or read out by the electronic unit, the respective data or information is directly and/or entirely processed by the at least one processor 72 of the electronic unit 70.

The electronic unit 70 (in particular, the communication unit 79) may be equipped with two different communication interfaces, namely with the short-range communication interface 76 and with the NFC interface 78. The short-range communication interface 76 is particularly configured to communicate with the external electronic device 80. The NFC interface 78 is particularly configured to capture or to read out data or information from the identifier 50.

The external electronic device 80 may be readily equipped with two different wireless communication interfaces, namely with the short-range communication interface 86, e.g., implemented as a Bluetooth or Wi-Fi interface to communicate with the short-range communication interface 76 of the electronic unit 70. The external electronic device 80 may be readily equipped with a further communication interface implemented as an NFC interface 84 to provide reading or capturing of data or information as provided by the identifier 50.

When implemented as a passive electronic identifier 52, e.g., as an NFC 53 tag, the external electronic device 80 and/or the electronic unit 70 may be brought in close vicinity to the identifier 50, which may be located inside the distal housing component 12 of the injection device 10. The transmission range of the NFC interfaces 78, 84 may be a few centimeters. Upon establishing a communication link, e.g., upon reading of the passive electronic identifier 52 by any of the NFC interfaces 78, 84, the electronic unit and/or the external electronic device 80 may provide an acoustic or visual confirmation to the user. This way, e.g., by the display 81 and/or by the speaker 82 the user may be informed that the electronic identifier 52 has been successfully read out.

The external electronic device 80 may be wirelessly paired to the electronic unit 70. Upon successful pairing or transferring of data or information of the identifier 50 between the electronic unit 70 and the external electronic device another visual and/or audible confirmation may be provided to the user, e.g., by the electronic unit 70 and/or by the external electronic device 80.

If approval or processing of the data or information of the identifier 50 reveals that the medicament container 30 is intended for use with the injection device 10, the at least one processor 72 is operable to deactivate the interlock 90, thereby allowing to set and/or to dispense or inject a dose of the medicament.

The at least one processor 72 and/or the at least one interlock 90, 91, 92, 93 may be implemented in various different ways. In one approach, the interlock 90 or locking mechanism may be operable to lock setting of a dose, to lock dispensing of a dose order to lock both, setting and dispensing of a dose.

The interlock 90 and/or the at least one processor 72 may be further configured to implement an automatic re-locking, e.g., after termination of each dispensing or in injecting procedure. Based on signals obtainable from the sensor 74, the at least one processor 72 may be provided with information being indicative of the remaining filling level of the medicament container 30. When the sensor 74 provides respective sensor signals that the medicament container 30 is substantially depleted or empty the at least one processor 72 may invoke activation of the interlock 92. Removal of the medicament container 30 or insertion of the medicament container 30 as well as disconnection or reconnection of the housing components 12, 14 may trigger an automated locking or disabling of the function of the drive mechanism for setting and/or for dispensing of a dose.

Information provided by the identifier 50 and capturable or readable by any of the communication unit 79, the NFC interface 78 and the wireless reader interface 85 may also include a counterfeit protection. A unique code should be verified, either by the electronic unit 70 and/or by a data exchange or data approval with the external database 96.

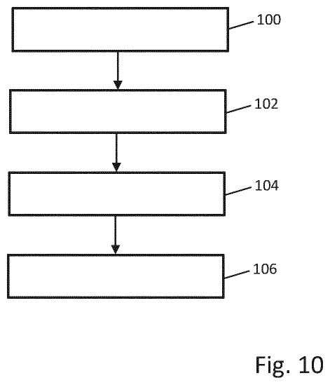

In the example illustrated in FIG. 10, at step 100, a user may bring the medicament container 30 provided with the identifier 50 in close vicinity to the electronic unit 70 and hence in close proximity to the NFC interface 78 so that the identifier 50 is within the transmission range of the NFC interface 78. This arrangement may be conducted manually, e.g., by a user of the injection device 10, before assembling the medicament container 30 in the distal housing component 12. Alternatively, if the medicament container 30 is preassembled inside the distal housing component 12, the distal housing component 12 together with the medicament container 30 and provided with the identifier 50 is brought in close proximity to the NFC interface 78 before the distal housing component 12 is connected to the proximal housing component 14.

In a subsequent step 102, the NFC interface 78 captures or reads the identifier 50 as provided on one of the distal housing component 12 and the medicament container 30.

In a further step 104 the respective information is validated by the at least one processor 72 of the electronic unit 70. The at least one processor 72 performs a data check and evaluates or processes the data obtained from the NFC interface 78. In case that the medicament container 30 is intended for use with this particular injection device 10 the respective data is approved and in step 106 the interlock 90 is controlled or operated by the at least one processor 72 to unlock the drive mechanism 8 of the injection device. A user may then commence to use the injection device 10 for setting of a dose and/or for injecting of a dose of the medicament 4. If the medicament container 30 is not intended for use with the injection device 10, the interlock 90 is triggered to block or to lock the drive mechanism 8 of the injection device 10.

### REFERENCE NUMBERS

1 drug delivery system
2 distal direction
3 proximal direction
4 medicament
6 protective cap
7 movable component
8 drive mechanism
10 injection device
11 housing
12 housing component
13 sidewall
14 housing component
15 aperture
16 connecting end
17 insert portion
18 flange
19 fastening element
20 connecting end
21 mount
22 through opening
23 needle assembly
24 needle hub
25 injection needle
26 window
27 receptacle
28 connecting end
30 container
31 barrel
32 sidewall
34 stopper
35 seal
36 cap
40 piston rod
41 pressure piece
42 housing insert
43 number sleeve
44 driver
45 clutch
46 dose dial
47 clutch
48 trigger
50 identifier
51 substrate
52 electronic identifier
53 NFC tag
54 optical identifier
55 antenna
57 memory
56 chip
58 code
70 electronic unit
71 printed circuit board
72 processor
73 battery
74 sensor
75 storage
76 short range communication interface
78 near field communication interface
79 communication unit
80 external electronic device
81 display
82 speaker
83 camera
84 near field communication interface
85 wireless reader interface
86 short range communication interface
88 wide range communication interface
90 interlock
91 interlock
92 interlock
93 interlock
94 network
96 database

1-19. (canceled)

20. An injection device for injecting of a dose of a medicament, the injection device comprising:
a housing configured to accommodate a medicament container,
a drive mechanism operably engageable with the medicament container for expelling or withdrawing the dose of the medicament from the medicament container, wherein the drive mechanism comprises at least one interlock configured to disable at least one of setting of the dose or dispensing of the dose,
an identifier provided on or associated with the medicament container,
an electronic unit operable to control the at least one interlock and comprising a near-field communication interface for obtaining information indicative of the identifier, wherein the electronic unit is configured to process signals of the near-field communication interface to at least temporarily deactivate the at least one interlock.

21. The injection device according to claim 20, wherein the housing comprises a distal housing component and a proximal housing component, wherein the distal housing component is configured to accommodate the medicament container and the proximal housing component is configured to accommodate or to support the drive mechanism.

22. The injection device according to claim 21, wherein the identifier is located on or inside the distal housing component and the electronic unit is provided in or on the proximal housing component.

23. The injection device according to claim 20, wherein the identifier comprises a passive electronic identifier.

24. The injection device according to claim 23, wherein the passive electronic identifier comprises a near-field communication (NFC) tag configured to communicate with the near-field communication interface of the electronic unit.

25. The injection device according to claim 20, wherein the electronic identifier comprises a non-volatile memory to store at least one of a medicament name, a medicament manufacturing date, a medicament expiry date, a lot number of the medicament, and a unique medicament container ID.

26. The injection device according to claim 25, wherein the non-volatile memory is erasable and/or writable by the near-field communication interface.

27. The injection device according to claim 20, wherein the interlock remains activated and use of the injection device is impeded or blocked when the identifier does not match with the drive mechanism and/or when the information obtained by the near-field communication interface does not match with predefined medicament container-related data.

28. The injection device according to claim 20, wherein a transmission range of the near-field communication interface is smaller than a spatial distance between the near-field communication interface and the identifier when the medicament container is operably engaged with the drive mechanism.

29. The injection device according to claim 20, wherein the electronic unit further comprises a short-range communication interface to communicate with a corresponding short-range communication interface of an external electronic device.

30. The injection device according to claim 29, wherein the transmission range of the near-field communication interface is smaller than a transmission range of the short-range communication interface.

31. The injection device according to claim 29, wherein the short-range communication interface comprises a wireless communication interface based on one of the following communication standards: Bluetooth, Bluetooth low energy (BLE), Zigbee or Wi-Fi.

32. The injection device according to claim 20, wherein the electronic unit comprises at least one processor operable to process data or information of the identifier captured or read out by the electronic unit.

33. The injection device according to claim 20, wherein:
the at least one interlock comprises an electromechanical interlock configured to mechanically engage with a first component of the injection device and with a second component of the injection device,
the first component is movable relative to the second component for at least one of setting of the dose and injecting of the dose, and
the at least one interlock is operable to switch between an interlocked state in which the first component is fixed relative to the second component and a release state in which the first component is movable relative to the second component.

34. The injection device according to claim 20, wherein:
the drive mechanism comprises at least one clutch operable to switch the drive mechanism between a dose setting mode and a dose injecting mode, and
the at least one interlock is operably engaged with the at least one clutch to selectively disable switching of the drive mechanism from one of the dose setting mode and the dose injecting mode into the other one of the dose setting mode and the dose injecting mode.

35. The injection device according to claim 20, wherein the medicament container filled with the medicament is arranged inside the housing.

36. A method of unlocking operation of an injection device for at least one of setting or injecting of a dose of a medicament, wherein:
the injection device comprises a housing configured to accommodate a medicament container and a drive mechanism operably engageable with the medicament container for expelling or withdrawing the dose of the medicament from the medicament container, and
the drive mechanism comprises at least one interlock configured to disable at least one of setting of the dose or dispensing of the dose, the method comprising:
capturing or reading an identifier provided on or associated with the medicament container by a near-field communication interface of an electronic unit of the injection device,
validating the identifier by comparing medicament-related data obtained from the identifier with predefined data related to the injection device, and
temporarily deactivating or re-activating the at least one interlock on the basis of the validation of the identifier.

37. The method according to claim 36, wherein for capturing or reading the identifier, the medicament container is at least temporarily arranged or brought within a transmission range of the near-field communication interface.

38. The method according to claim 36, wherein the electronic unit is configured to process signals of the near-field communication interface to at least temporarily deactivate the at least one interlock.

39. A computer-readable medium comprising instructions that, when executed by an electronic unit of an injection device, cause the electronic unit to execute the steps of:
capturing or reading an identifier provided on or associated with a medicament container of the injection device by a near-field communication interface of the electronic unit of the injection device,
validating the identifier by comparing medicament-related data obtained from the identifier with predefined data related to the injection device, and
at least one of temporarily deactivating or re-activating the at least one interlock on the basis of the validation of the identifier.
